(19) United States
(12) Patent Application Publication
(10) Pub. No.: US 2007/0087320 A1
(43) Pub. Date: Apr. 19, 2007

(54) Medium for conservation of organs, biological tissues or living cells

(76) Inventors: Daniel Licari, Grezieu Le Marche (FR); Eve Berthault, Grezieu Le Marche (FR)

Correspondence Address: CLARK & BRODY, 1090 VERMONT AVENUE, NW, SUITE 250, WASHINGTON, DC, 20005, US

(21) Appl. No.: 10/562,902

(22) PCT Filed: Mar. 23, 2004
(86) PCT No.: PCT/FR04/00712

§ 371 Date: Nov. 22, 2006

(30) Foreign Application Priority Data
Jul. 4, 2003 (FR) 03/08206

(51) Int. Cl. A01N 1/02; C12N 5/08

(52) U.S. Cl. 435/1.1; 435/368

(57) ABSTRACT

The object of the present invention is a preservation medium for living organs, biological tissues, and cells containing a liquid nutritive base, wherein it contains a high-molecular-weight hyaluronic acid and sodium chloride and wherein it contains no component of animal origin.

The present invention relates to the technical field of the preservation of living cells. More precisely, the object of the present invention is a new medium for preserving living organs, biological tissues, and cells, in particular living human corneas.

In the field of organ transplantation, when an organ is taken from a donor for the purpose of transplantation into a recipient, a preservation medium for the organ is required that is capable of maintaining the organ's viability in order for the transplant to succeed. In fact, a variety of media are often necessary. In the case of human corneas in particular, the media typically used include:
a transportation medium for the transfer of the corneas from the donation facility to the culture facility as well as for the transfer of the corneas from the culture facility to the transplantation facility,
a preservation medium. In general, preservation takes place at 4° C. or 31° C. This medium must guarantee optimal preservation of cellular viability in the medium term, that is to say approximately 4 to 5 weeks, maximum security in terms of quality (endothelial testing) and sterility (bacteriological, serological, and virological testing), and
a deturgescence medium, used approximately 24 hours before the transplant, in order to reduce the thickness of the cornea and to render it transparent.

The majority of the media used currently contain components of animal origin: fetal calf serum albumin, protein of animal origin such as transferrin, insulin, etc.

Due to the presence of components of animal origin in these media, it is difficult to guarantee their medical security with respect to prion diseases, in particular Creutzfeld-Jakob disease. Moreover, these media are susceptible to contamination by infectious agents and do not have a perfectly reproducible composition.

In this context, the present invention aims to provide a new preservation medium that preserves the viability of living cells.

Another objective of the invention is to provide a preservation medium that is inexpensive to manufacture by virtue of the components which it contains.

The preservation medium according to the present invention must also present maximum security in terms of quality and sterility.

Moreover, it presents the advantage of being capable of being prepared from components that are entirely synthetic, that is to say, that result from recombinant and chemical synthesis, and thus are nonimmunogenic and not contaminated by infectious agents. Consequently, the preservation medium according to the invention can present a precise composition that is reproducible from batch to batch.

More precisely, the invention relates to a preservation medium for living organs, biological tissues, and cells containing a liquid nutritive base, wherein it contains a hyaluronic acid of high molecular weight and sodium chloride, and wherein it contains no component of animal origin.

The invention also has as an aim the use of such a medium for the preservation, organ culture, cellular culture, transportation, and deturgescence of living organs, biological tissues, and cells, in particular living human corneas.

By living biological organs, cells, and tissues is meant components of human or animal origin including living fibroblasts, endothelial cells, and/or epithelial cells.

The preservation medium of the invention can be described as an ancillary therapeutic product.

The preservation medium according to the invention contains viscoelastic substances (VES) intended to protect the endothelial cells and surrounding tissues. These viscoelastic substances are in particular hyaluronic acid of high molecular weight.

Hyaluronic acid of high molecular weight, that is to say of molecular weight greater than or equal to 1 million daltons, can be of animal origin, condyloma acuminatum extract or umbilical cord blood, of bacterial origin (from cultures of streptococci), or of vegetable origin. Of course, the preservation medium according to the invention contains high-molecular-weight hyaluronic acid of vegetable origin, given that it is free of components of animal origin. In particular, the preservation medium of the invention will be prepared using high-molecular-weight hyaluronic acid from wheat, in powder form sold under the trade name Cristalhyal or in the form of a 1% aqueous solution sold under the trade name Vitalhyal, both from Laboratoire Bomann (Groupe Soliance), having a molecular weight equal to or greater than 10⁶ daltons and a Brookfield viscosity at 20° C. of 1,500 centipoises.

The preservation medium according to the invention also contains sodium chloride, as a crystalloid. The function of sodium chloride is, in particular, to avoid precipitation of the hyaluronic acid, but also to take part in the maintenance of osmolarity.

In particular, the preservation medium according to the invention contains:
from 80 to 4,000 mg/l, preferably 100 to 200 mg/l, preferentially from 100 to 160 mg/l of high-molecular-weight hyaluronic acid, and
from 4,500 to 9,000 mg/l, preferably from 5,500 to 9,000 mg/l, preferentially 7,000 mg/l of sodium chloride.

Advantageously, the medium according to the invention will contain poloxamer 188 which, in particular, has as a function to increase the viscosity of the medium.

Poloxamer 188, also called Pluronic F68 and Lutrol® F68, is a polyoxyethylene-polyoxypropylene polymer sequence of molecular weight 7,680-950 g/mol and of general formula:
HO—(CH₂—CH₂—O)ₓ—[CH₂—CH(CH₃)—O]_{y}—(CH₂—CH₂—O)ₓ—H

where x is approximately equal to 79 and y approximately equal to 28.

The presence of poloxamer 188 is particularly advantageous in the medium according to the invention when said medium is intended to be used for the deturgescence of organs and the transport and preservation of living tissues, cells, and, in particular, human cornea transplants. The medium according to the invention will contain, preferably, from 200 to 75,000 mg/l, preferentially from 450 to 50,000 mg/l of poloxamer 188.

The preservation media currently on the market intended for the deturgescence of corneas contain dextran. The function of dextran is to decrease the thickness of the cornea and could be used in the media according to the invention intended for the deturgescence of corneas. Nevertheless, poloxamer 188, which too decreases the thickness of the cornea but which is much less cytotoxic, is preferred over dextran.

Methyl cellulose is another VES which the preservation medium according to the invention can contain. Methyl cellulose is of vegetable origin and is obtained from cellulose fibers from cotton flock or wood pulp. These cellulose fibers are treated with a caustic soda solution in order to undergo etherification with methylene chloride. The degree of substitution, corresponding to the number of methoxylated substituents per glucoside unit, is between 1.64 and 1.92. In particular, to prepare the preservation medium of the invention, methyl cellulose marketed by SEPPIC under the trade name Metolose SM 400 with a Brookfield viscosity of 4,000 centipoises (2% in water at 20° C.) and a molecular weight of 86,000 daltons can be used. The medium according to the invention will contain, preferably, from 210 to 5,000 mg/l, preferably from 1,900 to 2,500 mg/l, preferentially 2,205 mg/l of methyl cellulose.

The VES used enable cell hydration by the retention of water and present a certain adhesiveness or attachment to the cells and tissues that they surround, thus ensuring the protection of said surrounding cells and tissues against chemical attacks and the toxic effects of air.

The preservation medium according to the invention also contains other components more typically used in the field of living-cell preservation.

In particular, the preservation medium contains an aqueous chemical nutritive base classically used in organ and cell culture preservation media. A notable reference article is “Le technoscope de biofutur”, no. 133, April 1994, pages 3-16, which indicates that a nutritive base contains:
amino acids, whose role in cellular metabolism is to provide nitrogen and carbon. Certain cells have specific needs in addition to the 13 essential amino acids (serine, for example, for lymphoid cells);
sugars, of which glucose is most widely used although it can be replaced by galactose when it is necessary to limit the accumulation of lactic acid;
vitamins, primarily group B, of which 8 are regarded as essential;
ions, supplied in the form of balanced saline solutions, which play an important part in the maintenance of membrane potential and osmotic pressure and are also cofactors for many enzymatic reactions;
trace metals, which appear to play an increasingly important role, in particular when the culture is carried out in a precise medium. The most important are selenium, cadmium, and lithium.

This type of base can be prepared from these various constitutive elements. Certain chemical nutritive bases are also available commercially in both liquid and solid form: the latter must then be reconstituted in water. Of use in particular are IMDM (Iscove's modified Dulbecco's medium, ref.: Iscove, N. N. and Melchers (1978) J. of Exp. Med. 147: 923-933), MEM Alpha from Stainers, C. P. et al., Nature New Biol. 230, 52 (1971), Click RPMI from Click et al., Cell. Immunol. 3, 264 (1972), CMRL1066 medium from Parker, R. C., et al., Special Publications, N.Y. Academy of Sciences, 5, 303, (1957), Leibovitz L15 medium from Leibovitz, A., Am. J. Hyg. 78, 173 (1963) and Morton, H. J., In vitro, 6, 89 (1970), M199 medium from Morgan, J. F et al., Proc. Soc. Exp. Biol. Med. 73, 1 (1950), and DMEM/HAM F12 medium from Barnes, D. and Sato, G., Anal. Biochem. 102, 255 (1980), aqueous nutritive bases which contain various substances necessary for the maintenance of cells and tissues, in particular various trace elements, amino acids, vitamins, electrolytes, a stabilizing pH buffer, a pH indicator (phenol red, for example), and glucose or galactose (L15). By trace elements is meant all metallic inorganic salts, except for NaCl, present in trace amounts or in larger amounts.

In an advantageous way, the preservation medium according to the invention thus contains amino acids, trace elements, vitamins, electrolytes, and a stabilizing pH buffer, supplied primarily by the nutritive base used. If the base used does not contain these elements in sufficient quantity, said elements will be supplemented.

The preservation medium according to the invention contains, advantageously and independently, from 1 to 50 mg/l of chondroitin sulfate, from 0.1 to 25 mg/l of heparan sulfate, from 500 to 2,000 mg/l of alginic acid, and from 1,000 to 10,000 mg/l of hetastarch.

In the case where the preservation medium is intended to be used for human cornea transplants, it will by preference contain components present in the aqueous humor such as sodium lactate, sodium acetate, sodium citrate, iron (II) ascorbate, iron (II) gluconate, sodium pyruvate, and calcium chloride.

In a particularly advantageous way, the preservation medium according to the invention contains independently or in combination:
from 0.01 to 350 mg/l of vitamins, preferably selected from among:
   tocopherol acetate
   retinol acetate
   hydroquinone
   ascorbic acid
   thiamin B1-HCL
   riboflavin B2
   calcium D-pantothenate B5
   pyridoxal HCl B6
   biotin B8
   folic acid B9
   cyancobalamine B12
   nicotinamide B3 PP
   chromium orotate B13
from 0.01 to 650 mg/l of trace elements, preferably selected from among:
   CaCl₂, 2H₂O
   KCl
   CaH₂PO₄.2H₂O
   NaH₂PO₄.H₂O
   NaHCO₃
   MgCO₃.7H₂O
   MgSO₄.7H₂O
   FeSO₄.7H₂O
   CuSO₄.5H₂O
   MnCO₃.4H₂O
   MnCl₂.4H₂O
   Na₂SiO₃.9H₂O
   H₂SeO₃
   NH₄VO₃
   (NH₄)6Mo₇O₂₄.4H₂O
   SnCl₂.2H₂O
   ZnSO₄.7H₂O
   zinc oxide
   NiCl₂.6H₂O
from 0.005 to 150 mg/l of nucleosides, preferably selected from among:
   adenosine
   cytidine
   deoxyadenosine
   deoxycytidine
   deoxyguanosine
   guanosine
   uridine
   thymidine
from 800 to 4,000 mg/l of amino acids,
from 500 to 9,000 mg/l of monosaccharides, and preferably of glucose and/or galactose,
other elements, at a total concentration 0.001 to 75,000 mg/l, and in particular:
   sodium acetate (3H₂O)
   sodium citrate
   sodium lactate
   sodium pyruvates
   iron (II) gluconate
   sodium selenite
   poloxamer 188
   oleic acid
   linoleic acid
   linolenic acid
   palmitic acid
   Tween 80.

The preservation medium according to the invention can be in liquid or semi-solid form. It presents a viscosity that is significant enough to support cell protection. In an advantageous way, the Brookfield viscosity of the preservation medium according to the invention is in the range between 1 and 15 centipoises (cps) at 20° C., preferably between 2.5 and 10 cps. The preservation medium according to the invention is thus non-injectable by virtue of its viscosity.

The osmolarity of the medium according to the invention is also important and, in particular, is in the range between 300 and 465 mOsm±40. The osmolarity of the medium depends in particular on the NaCl concentration. When the medium according to the invention is intended for preservation or transport, its osmolarity advantageously will be in the range between 300 and 360 mOsm±40; when it is intended for deturgescence, its osmolarity advantageously lies between 350 and 465 mOsm±40. The osmolarity of the preservation media currently on the market is lower. One of the advantages of the invention is to be able to provide a single medium for transport, preservation, and deturgescence.

The preservation medium according to the invention is prepared by mixing the various components. Preferably, to improve dissolution of the hyaluronic acid in the liquid biological nutritive base, the latter will be mixed with sodium chloride then added to the nutritive base already containing methyl cellulose.

The preservation medium according to the invention contains no components of animal origin. Indeed, on one hand, contrary to the majority of the media used to date for the preservation of living organs, biological tissues, and living cells, the medium according to the invention contains neither fetal calf serum albumin, nor lactoferrin, nor transferrin, nor insulin, nor other proteins of animal origin. In addition, high-molecular-weight hyaluronic acid and methyl cellulose, whose synthesis utilizes no raw material of animal origin, are used. Such a preservation medium could thus easily be in conformity with the legislation on ancillary therapeutic products defined in article L. 1263-1 of the French Public Health Code. The use of a preservation medium free of components of animal origin makes it possible to improve the medical security of the preserved cells.

The preservation medium according to the invention could be used for the preservation, organ culture, cellular culture, freezing, transport, and deturgescence of living organs, biological tissues, and cells, and in particular living human corneas. The preservation medium according to the invention is usable at temperatures ranging between −196° C. and 37° C. in particular.

The preservation medium according to the invention may be adapted according to the application envisaged.

For example, if the medium according to the invention is intended to be used for preoperative deturgescence, it will advantageously contain poloxamer 188 at a concentration of 200 to 75,000 mg/l, preferentially from 450 to 50,000 mg/l.

If the medium is intended to be used for the freezing of living cells, a portion of the water present in the medium could be replaced by dimethyl sulfoxide (DMSO) which has a cryoprotective effect.

The following examples illustrate the invention, but are in no way restrictive. The following examples use hyaluronic acid in powder form sold under the trade name Cristalhyal by Laboratoire Bowman (Groupe Soliance), methyl cellulose sold by SEPPIC under the trade name Metolose SM 4000, and NaCl marketed by Sigma Aldrich. Examples 1 to 3 (preservation media) have an IMDM base volume of 74% and examples 4 to 7 (deturgescence media) have an IMDM base volume of 88%.

Osmolarities are measured with an osmometer sold by Fisher Bioblock Scientific under the reference M85501 (automatic zero [distilled water] and standard [300 mOsm/kg] calibration by pressing a key; response time 1 minute).

Viscosities are measured with a viscometer sold by Fisher Bioblock Scientific under the reference M57571 with a low-viscosity adapter starting at 1 cps ref. M57510 (simultaneous display of speed, selected mobile phase, viscosity in cps and in % of range, and temperature; Brookfield compatible; the mobile phases are plunged directly in the sample).

### EXAMPLE 1

### The Example 1 Medium is Advantageously Used for Transport and Preservation

| | | |
|---|---|---|
| High-molecular-weight | 100 | mg/l |
| hyaluronic acid | | |
| Methyl cellulose | 2,205 | mg/l |
| NaCl | 6,985 | mg/l |
| Amino acids | 1,838 | mg/l |
| Trace elements | 5,390 | mg/l |
| Glucose | 4,500 | mg/l |
| Carbohydrates | 1,167 | mg/l |
| Nucleosides | 10 | mg/l |
| Vitamins | 163 | mg/l |
| Fatty acid esters | 46 | mg/l |
| Buffer | 8,982 | mg/l |
| pH | 7.3 | |
| Osmolarity | 394 | mOsm |
| Viscosity | 5 | cps (Brookfield, 20° C.) |

### EXAMPLE 2

### The Example 2 Medium is Advantageously Used for Transport and Preservation

| | | |
|---|---|---|
| High-molecular-weight | 100 | mg/l |
| hyaluronic acid | | |
| Methyl cellulose | 2,205 | mg/l |
| NaCl | 5,585 | mg/l |
| Amino acids | 1,838 | mg/l |
| Trace elements | 5,390 | mg/l |
| Glucose | 4,500 | mg/l |
| Carbohydrates | 1,167 | mg/l |
| Nucleosides | 10 | mg/l |
| Vitamins | 163 | mg/l |
| Fatty acid esters | 46 | mg/l |
| Buffer | 8,982 | mg/l |
| pH | 7.3 | |
| Osmolarity | 372 | mOsm |
| Viscosity | 5 | cps (Brookfield, 20° C.) |

### EXAMPLE 3

### The Example 3 Medium is Advantageously Used for Transport and Preservation

| | | |
|---|---|---|
| High-molecular-weight | 160 | mg/l |
| hyaluronic acid | | |
| Poloxamer 188 | 2,205 | mg/l |
| NaCl | 5,585 | mg/l |
| Amino acids | 1,838 | mg/l |
| Trace elements | 5,390 | mg/l |
| Glucose | 4,500 | mg/l |
| Carbohydrates | 1,167 | mg/l |
| Nucleosides | 10 | mg/l |
| Vitamins | 163 | mg/l |
| Fatty acid esters | 46 | mg/l |
| Buffer | 8,982 | mg/l |
| pH | 7.3 | |
| Osmolarity | 305 | mOsm |
| Viscosity | 1.5 | cps (Brookfield, 20° C.) |

### EXAMPLE 4

### The Example 4 Medium is Advantageously Used for Deturgescence

| | | |
|---|---|---|
| High-molecular-weight | 100 | mg/l |
| hyaluronic acid | | |
| Methyl cellulose | 2,205 | mg/l |
| Dextran | 50,000 | mg/l |
| NaCl | 6,895 | mg/l |
| Amino acids | 1,838 | mg/l |
| Trace elements | 5,390 | mg/l |
| Glucose | 4,500 | mg/l |
| Carbohydrates | 1,167 | mg/l |
| Nucleosides | 10 | mg/l |
| Vitamins | 163 | mg/l |
| Fatty acid esters | 46 | mg/l |
| Buffer | 8,982 | mg/l |
| pH | 7.3 | |
| Osmolarity | 694 | mOsm |
| Viscosity | 10 | cps (Brookfield, 20° C.) |

### EXAMPLE 5

### The Example 5 Medium is Advantageously Used for Deturgescence

| | | |
|---|---|---|
| High-molecular-weight | 100 | mg/l |
| hyaluronic acid | | |
| Methyl cellulose | 2,205 | mg/l |
| Dextran | 50,000 | mg/l |
| NaCl | 5,585 | mg/l |
| Amino acids | 1,838 | mg/l |
| Trace elements | 5,390 | mg/l |
| Glucose | 4,500 | mg/l |
| Carbohydrates | 1,167 | mg/l |
| Nucleosides | 10 | mg/l |
| Vitamins | 163 | mg/l |
| Fatty acid esters | 46 | mg/l |
| Buffer | 8,982 | mg/l |
| pH | 7.3 | |
| Osmolarity | 585 | mOsm |
| Viscosity | 10 | cps (Brookfield, 20° C.) |

### EXAMPLE 6

### The Example 6 Medium is Advantageously Used for Deturgescence

| | | |
|---|---|---|
| High-molecular-weight | 160 | mg/l |
| hyaluronic acid | | |
| Dextran | 50,000 | mg/l |
| NaCl | 5,585 | mg/l |
| Amino acids | 1,838 | mg/l |
| Trace elements | 5,390 | mg/l |
| Glucose | 4,500 | mg/l |
| Carbohydrates | 1,167 | mg/l |
| Nucleosides | 10 | mg/l |
| Vitamins | 163 | mg/l |
| Fatty acid esters | 46 | mg/l |
| Buffer | 8,982 | mg/l |
| pH | 7.3 | |
| Osmolarity | 595 | mOsm |
| Viscosity | 8.5 | cps (Brookfield, 20° C.) |

### EXAMPLE 7

### The Example 7 Medium is Advantageously Used for Deturgescence

| | | |
|---|---|---|
| High-molecular-weight | 160 | mg/l |
| hyaluronic acid | | |
| Poloxamer 188 | 50,000 | mg/l |
| NaCl | 5,585 | mg/l |
| Amino acids | 1,838 | mg/l |
| Trace elements | 5,390 | mg/l |
| Glucose | 4,500 | mg/l |
| Carbohydrates | 1,167 | mg/l |
| Nucleosides | 10 | mg/l |
| Vitamins | 163 | mg/l |
| Fatty acid esters | 46 | mg/l |
| Buffer | 8,982 | mg/l |
| pH | 7.3 | |
| Osmolarity | 376 | mOsm |
| Viscosity | 5 | cps (Brookfield, 20° C.) |

Materials and Methods
The Preservation Sequence

The scientific human corneas (organs donated to science) are taken in the 24 hours following the death of the donor. The donors should not have undergone intraocular surgery in order to maintain comparability of the two corneas from the same donor. Following removal, each cornea of the pair is immersed in 50 ml of a transport medium. Either a reference medium (Inosol®, Chauvin-Opsia/Baush and Lomb, Toulouse, France) or a medium according to the invention (examples 1 to 3) is used. The sealed flasks containing the corneas are immediately placed in a drying oven at 31° C. On the second day of preservation in organ culture, the endothelial cell density (ECD) is measured according to a procedure described below. The cornea is then re-submerged in the same type of medium in a new 100 ml flask and is suspended with a suture thread in order to avoid contact with the walls of the flask and with the sediments deposited at the bottom of the flask. After 14 days of preservation, the corneas are transferred to new 100 ml flasks. After 30 days of preservation, the maximum recommended period in Europe, a new ECD measurement is performed and the cell loss is calculated for the given period of preservation. The cornea is then immersed in 50 ml of a “deturgescence” medium intended to reduce its thickness. Either Exosol® (Chauvin-Opsia/Baush and Lomb) or a medium according to the invention (examples 4 to 7) corresponding to 50,000 mg/l of dextran or 50,000 mg/l of Poloxamer 188 is used. Forty-eight hours later, the two corneas of the same pair are photographed positioned side-by-side on a back-lit grid of 8 black lines of increasing thickness. This photography serves as a record of corneal transparency. Corneal thickness is measured at the apex by ultrasonic pachymetry (Tomey AL-2000, Tokyo, Japan). The third ECD measurement is performed after incubation for 45 seconds with 4% alizarin red (Sigma) in pH 4.5 phosphate buffer for the purpose of coloring the cell membranes. This nonvital coloring can be used only at the end of preservation because of its cellular toxicity.

The entire procedure is performed blind concerning the nature of the preservation medium.

Procedure for Assuring Blind Analysis

The two preservation and deturgescence media are packaged in identical containers (125 ml Nalgene flasks) and are numbered by a person who neither takes part in preservation nor in ECD determinations. A numbering system based on a randomization table makes identification of the media according to the number on the flasks unlikely.

ECD Measurement Procedure

After rinsing the cornea with BSS (balanced salt solution, Alcon, Kaysersberg, France), the endothelium is covered for 1 minute with 0.4% trypan blue (Sigma) then rinsed for 4 minutes with 0.9% sodium chloride. The corneal endothelium is then observed at 10× under an optical microscope coupled with the prototype of the endothelial mosaic analyzer described by Gain P. et al. in Br J Ophthalmol 2002, 86, pages 306-11 and 531-6. Ten images of distinct areas of the endothelium are captured and archived on a computer hard disk for later analysis. Each analysis involves more than 300 cells.

Results

Donor Characteristics

The donors are 6 women and 10 men whose ages range between 57 and 90 years, with an average age of 74.4 years. The delay between death and removal of the corneas ranges between 4.5 to 44 hours, with an average delay of 20 hours.

Results

| | | Example 1 |
|---|---|---|
| | | (preservation) |
| | Opsia | Example 4 |
| | media | (deturgescence) |
| ECD at the beginning of preservation (d2) | 1814 | 1848 |
| ECD at the end of preservation (d30) | 1600 | 1693 |
| Cell loss (%) | −11.8 | −8.4 |
| Post-deturgescence ECD | 1300 | 1542 |
| Post-deturgescence cell loss (%) | −18.7 | −8.9 |
| Post-deturgescence corneal thickness (μm) | 703 | 717 |

Total loss in %: Opsia media: 30.5 and media from examples 1 and 4 according to the invention: 17.3

| | | Example 2 |
|---|---|---|
| | | (preservation) |
| | Opsia | Example 5 |
| | media | (deturgescence) |
| ECD at the beginning of preservation (d2) | 1373 | 1392 |
| ECD at the end of preservation (d30) | 1280 | 1300 |
| Cell loss (%) | −6.8 | −6.7 |
| Post-deturgescence ECD | 980 | 1163 |
| Post-deturgescence cell loss (%) | −23.4 | −10.5 |
| Post-deturgescence corneal thickness (μm) | 723 | 716 |

Total loss in %: Opsia media: 30.2 and media from examples 2 and 5 according to the invention: 17.2

| | Opsia | Example 3 |
|---|---|---|
| | media | Example 6 |
| ECD at the beginning of preservation (d2) | 2441 | 2190 |
| ECD at the end of preservation (d30) | 2239 | 2090 |
| Cell loss (%) | −8.3 | −4.6 |
| Post-deturgescence ECD | 1573 | 2255 |
| Post-deturgescence cell loss (%) | −29.7 | −3 |
| Post-deturgescence corneal thickness (μm) | 797 | 950 |

Total loss in %: Opsia media: 38 and media from examples 3 and 6 according 5 to the invention: 7.6

| | Opsia | Example 3 |
|---|---|---|
| | media | Example 7 |
| ECD at the beginning of preservation (d2) | 2788 | 2602 |
| ECD at the end of preservation (d30) | 2239 | 2370 |
| Cell loss (%) | −29.4 | −8.9 |
| Post-deturgescence ECD | 1928 | 2088 |
| Post-deturgescence cell loss (%) | −2.1 | −11.9 |
| Post-deturgescence corneal thickness (μm) | 755 | 832 |

Total loss in %: Opsia media: 31.5 and media from examples 3 and 7 according to the invention: 20.8

Discussion

With the conclusion of this study, the inventors have developed a precise medium free of any component of animal origin capable of ensuring significantly higher endothelial survival over 30 days than that obtained with the reference medium used in cornea banks. This point is of primary importance because an additional capital of endothelial cells of almost 16.9% is obtained on average, the result of which is a spectacular improvement in preservation quality. Such a gain would make it possible for the recipient to have a higher endothelial reserve than that which has been possible to date. For the recipient, this reserve means better resistance to intercurrent events (trauma, immunological rejections, endo-ocular surgery) and also an increase in the period during which the transplant remains transparent.

Poloxamer 188 appears less cytotoxic than dextran based on the results obtained.

1. Preservation medium for living organs, biological tissues, and cells containing a liquid nutritive base, wherein it contains a high-molecular-weight hyaluronic acid and sodium chloride and wherein it contains no component of animal origin.

2. Preservation medium according to claim 1 wherein it contains:
from 80 to 4,000 mg/l, preferably 100 to 200 mg/l, preferentially 100 to 160 mg/l of high-molecular-weight hyaluronic acid, and
from 4,500 to 9,000 mg/l, preferably from 5,500 to 9,000 mg/l, preferentially 7,000 mg/l of sodium chloride.

3. Preservation medium according to claim 1, wherein it contains, in addition, poloxamer 188.

4. Preservation medium according to claim 3, wherein it contains from 200 to 75,000 mg/l, preferably from 450 to 50,000 mg/l of poloxamer 188.

5. Preservation medium according to claim 1 wherein it contains, in addition, methyl cellulose.

6. Preservation medium according to claim 5, wherein it contains from 210 to 5,000 mg/l, preferably from 1,900 to 2,500 mg/l and preferentially 2,205 mg/l of methyl cellulose.

7. Preservation medium according to claim 1, wherein it presents an osmolarity from 300 to 465 mOsm±40 mOsm.

8. Preservation medium according to claim 1, wherein it presents a Brookfield viscosity at 20° C. in the range between 1 and 15 centipoises, preferably between 2.5 and 10 centipoises.

9. Preservation medium according to claim 1, wherein it contains trace elements, amino acids, vitamins, and a stabilizing pH buffer.

10. Preservation medium according to claim 1, wherein it does not contain dextran.

11. Use of a preservation medium according to claim 1 for the preservation of living human corneas.

12. Use of a preservation medium according to claim 1 for organ culture of living organs, biological tissues, and cells, in particular of living human corneas.

13. Use of a preservation medium according to claim 1 for the transport of living organs, biological tissues, and cells, in particular of living human corneas.

14. Use of a preservation medium according to claim 1 for the deturgescence of living organs, biological tissues, and cells, in particular of living human corneas
